(19) United States
(12) Patent Application Publication
(10) Pub. No.: US 2006/0015125 A1
(43) Pub. Date: Jan. 19, 2006

(54) Devices and methods for gastric surgery

(76) Inventor: Paul Swain, London (GB)

Correspondence Address: KNOBBE MARTENS OLSON & BEAR LLP, 2040 MAIN STREET, FOURTEENTH FLOOR, IRVINE, CA, 92614, US

(21) Appl. No.: 11/123,889

(22) Filed: May 6, 2005

Related U.S. Application Data
(60) Provisional application No. 60/568,929, filed on May 7, 2004.

(51) Int. Cl. A61B 17/08

(52) U.S. Cl. 606/151

(57) ABSTRACT

Disclosed are an intragastric support frame, for implantation within the stomach for therapeutic or diagnostic purposes. Also disclosed is a tissue anchor deployment system, for attachment to a tissue wall.

### CROSS-REFERENCE TO RELATED APPLICATIONS

This application claims priority under 35 U.S.C. § 119(e) from U.S. Provisional Application Ser. No. 60/568,929 filed May 7, 2004, the entirety of which is hereby incorporated by reference.

### BACKGROUND OF THE INVENTION

1. Field of the Invention

The present invention relates to devices and methods for performing gastric surgery, particularly for facilitating gastric surgery using endoscopic methods, as described below.

2. Description of the Related Art

Gastrointestinal sleeve devices for treatment of obesity have been described in prior applications, as have various devices and methods for attachment of a gastrointestinal sleeve device within a patient's digestive tract. The present invention is directed to soft intragastric frames that may be used to house various devices used during surgery. The present invention is also directed to new devices and methods for sewing through an endoscope.

### SUMMARY OF THE INVENTION

A tissue anchor deployment system, for advancing through a channel in an endoscope, comprising: a tubular body, having a sharpened distal end; a tissue attachment structure within the tubular body; and a removable sheath surrounding at least the sharpened distal end, for isolating the sharpened distal end from a wall of the channel.

An intragastric support frame or implantation in the stomach, comprising: at least a first and a second inflatable balloon, each having an elongate curved body with a proximal end and a distal end, at least a first and a second inflatable balloon connected together at each of the proximal and distal ends to form a support frame; wherein the fully assembled and inflated support frame is sufficiently dimensioned to prevent passage through the pyloris.

### BRIEF DESCRIPTION OF THE DRAWINGS

FIG. 1 shows an intragastric soft building frame in a convex-outward configuration.

FIG. 2 shows an intragastric soft building frame in a concave-outward hourglass configuration.

FIGS. 3A-3B and 4A-4E show devices and methods for sewing through a conventional endoscope.

### DETAILED DESCRIPTION OF THE PREFERRED EMBODIMENT

An intragastric soft building frame can be formed as a structure made out of balloons using three or four banana-shaped balloons connected at the top and bottom into a frame. They could be assembled together inside the stomach or could be pre-assembled and expanded inside the stomach to form the necessary shape. Suitable connectors can be provided on the appropriate surfaces of the balloons for assembling the building frame together in the desired configuration. The device could be used for parking objects for use during an operation, for example an endoscopic camera, surgical instruments or components, or implantable devices. Alternatively, it could be implanted inside the stomach for short or extended periods of time and could support other structures, which could process or conduct fluid or solid materials through the stomach. It could hold a camera for long-term use. The device provides a light, soft structure. The device could be inflated with a gas, such as air or helium, or with a liquid, such as saline solution. Mucosal contact points should be softened to avoid ischemia due to the weight of the device and any other structures attached to it. The size of the intragastric soft building frame relative to the stomach can be varied based upon on the clinical application and the anatomy of the individual patient. The expanded dimension should be large enough to prevent passage through the pylorus.

The banana-shaped balloons of the building frame can be assembled in a number of different configurations.

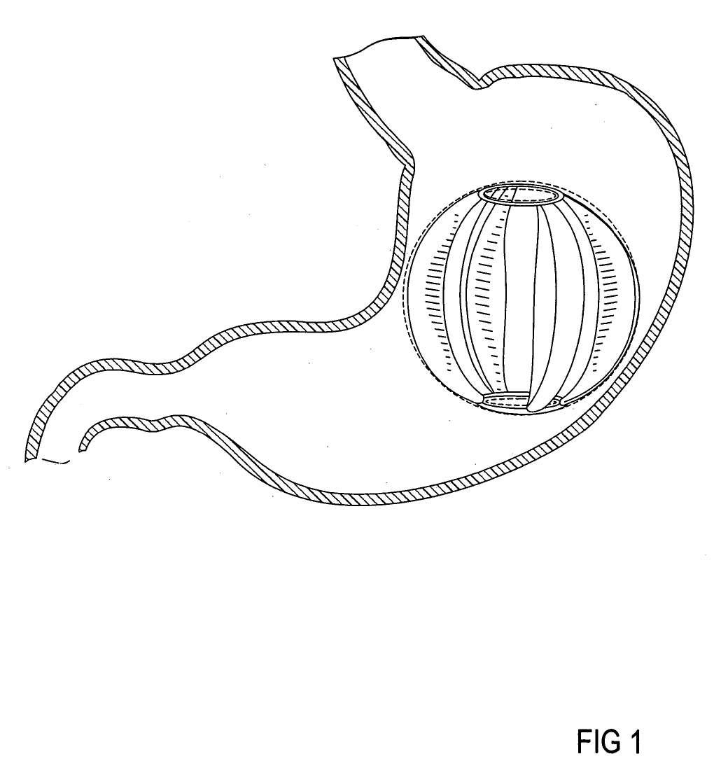

FIG. 1 shows the balloons assembled in a convex-outward hourglass configuration. Depending on the curvature, size and spacing of the balloons, the assembled configuration may look approximately like a football, a rugby ball or a soccer ball. This configuration of the building frame will be good for long term use, such as to control flow of food and liquids through the stomach because the rounded sides and ends will not place undue stress on the stomach walls. Optionally, the intragastric soft building frame may be constructed with a membrane connecting the assembled balloons. The extent and location of the membrane covering the building frame, as well as the size and spacing of the balloons, will control the resistance to flow through the stomach.

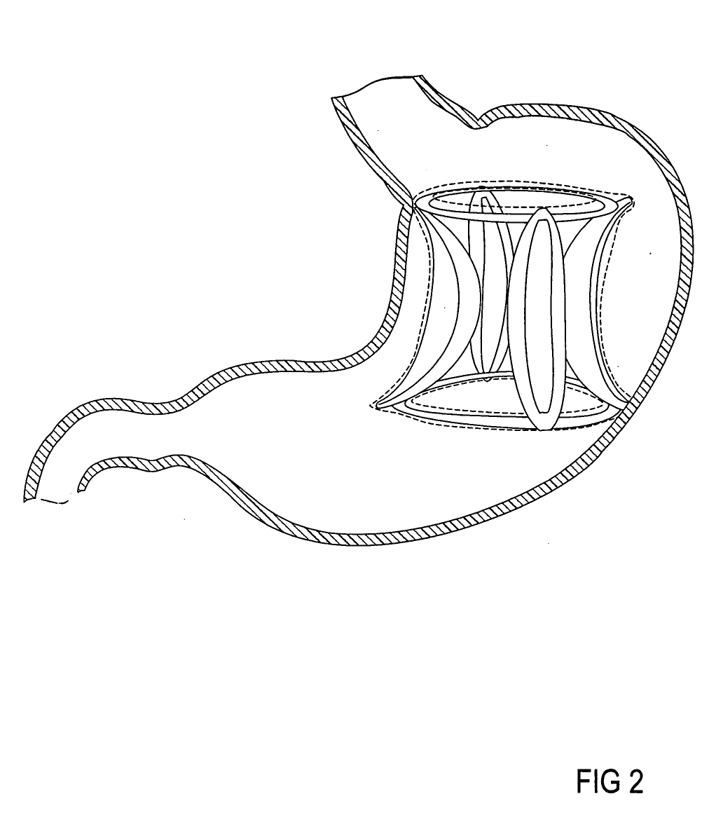

FIG. 2 shows the balloons assembled in a concave-outward hourglass configuration. In this configuration, the building frame will be stable and less likely to rotate within the stomach. Optionally, the intragastric soft building frame may be constructed with a membrane connecting the balloons around the outside, at the top, at the bottom and/or at some intermediate portion. A membrane across the top will make the building frame useful as an intraoperative tool rest, whereas a membrane across the bottom will make it more like a bucket for holding tools and components intraoperatively. When used to control flow of food and liquids through the stomach, the extent and location of the membrane covering the building frame, as well as the size and spacing of the balloons, will control the resistance to flow.

The inflatable balloons can be made of silicone, PU, PE, polyolefin, PET or other polymeric material. Materials such as PE and PET could be advantageous as they can be configured to have less distention, deflection and/or deformation and thereby provide improved mechanical support. Mechanical enhancements such as ribs, folds or reinforcing materials such as nylon or Kevlar fibers can also be included to enhance mechanical support. Balloon devices would preferably be inflated in place and would include inflating and/or deflating means. If the inflating/deflating means were removable, a reversible coupling means and/or a valve or inflation port sealing means could also be included. If used to support devices e.g. endoscopic sewing devices, mechanical coupling can optionally be included to interface with devices using the intragastric support. Examples of such couplings include U-shaped channels, rings, hooks, snaps and other means known in the art.

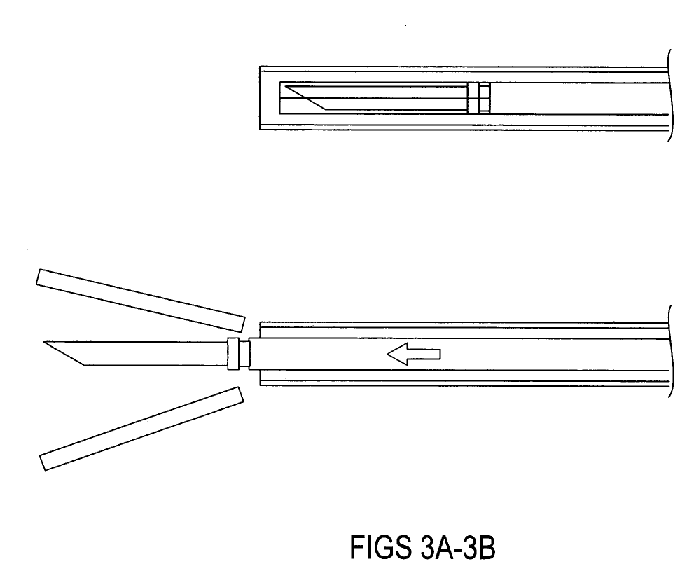

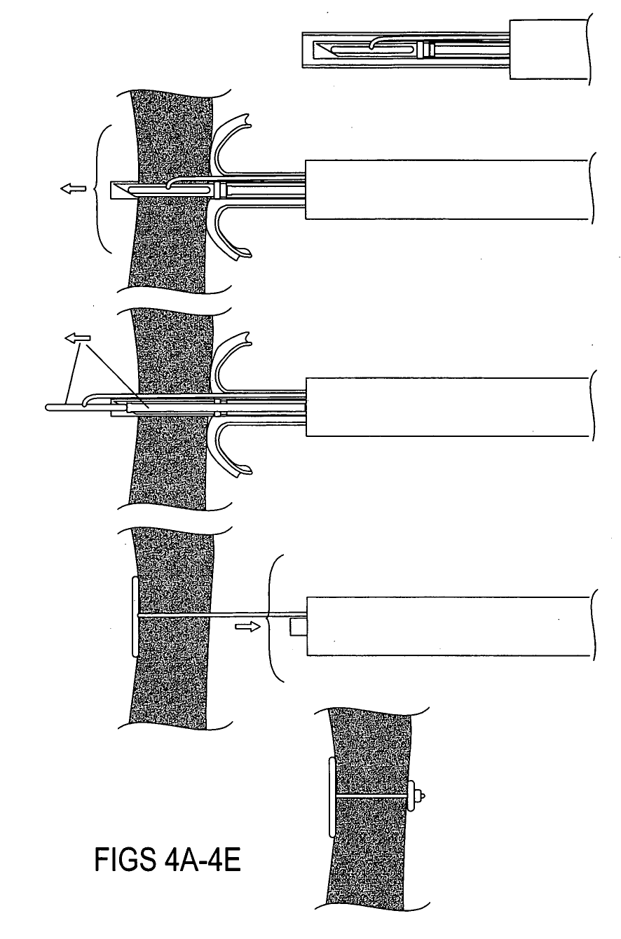

Devices and methods are described for sewing through the biopsy channel of a conventional endoscope, as shown in FIGS. 3-4. All the stages of sewing, cutting thread and tying knots or thread locking can be accomplished through a biopsy channel, optionally 2.8 mm or larger. The method does not require the use of endoscopic ultrasound (EUS), although it could be used with ultrasound real-time imaging to advantage to sew into specific organs or tissue depth.

Some endoscopic sewing methods use suction to control the depth of needle penetration into tissue. These include the BARD Endocinch and the Wilson Cook SewRight. These methods have two disadvantages. They increase the overall diameter of the endoscope from 11 mm to 15-18 mm depending on the size of the overtube or sewing capsule head. This makes the procedure uncomfortable for the patients and the procedure must be done under heavy sedation or general anesthesia. The other difficulty is that, when suction is applied to a cavity, the subsequent depth of gastric muscle penetrated by the needle is variable. This is in part due to the variable loose attachment of the mucosa and submucosa to the muscle and in part due to some variation in thickness of tissue. Another issue is that the tissue may be sucked into the cavity as two adjacent folds and the needle may run completely or partly between the folds thus failing to penetrate as deeply as is desirable. This seems to be due to large variations in stomach wall thickness, which is confirmed by measurements made of the stomach wall thickness in patients having resections for bleeding gastric ulcers (published in Gastroenterology in 1986). These measurements however were all performed on the wall adjacent to the ulcer, which was the point of interest for the study. Recent measurements of wall thickness with EUS at live surgery suggest that there may not be as much variation in wall thickness in healthy tissue. Nonetheless, wall thickness becomes a significant factor when it is important to sew to the correct depth using flexible endoscopy without knowledge of the gastric wall thickness. Pushing a needle into tissue tends to compress the mucosa and submucosa against the muscle, while suctioning the mucosa into a cavity tends to expand the distance to the serosa. Depending on the outer diameter of the needle and its sharpness and coefficient of friction, there may be some drag as the needle penetrates the tissue, which may increase the distance the needle must travel to penetrate to the serosa. The needle bevel is an important factor in the force required to push through tissue and will also influence the distance the T member of a T-tag fastener delivered through the needle must travel to reach its target. The distance of travel of the pushing rod may also need to be varied if the rod is to be used with the endoscope in both straight and extreme flexion configurations. The sewing method could be used with a T-tag fastener and suture as described herein. New knotting mechanisms and new ways of cutting thread are also disclosed. All of these can be deployed through a 2.8 mm diameter channel of a conventional gastroscope and do not require that the instrument be removed to tie knots or place extra stitches.

One goal of aspects of this invention is to develop new devices and methods for sewing during flexible endoscopy using sutures or fasteners, such as T-tag fasteners. The device includes a needle that can be pushed through tissue. There is an adjustable stop that allows penetration to a predetermined depth. The needle is short and is attached to a flexible shaft in order to allow the needle to be used in a flexed endoscope without restricting the bending radius of the scope, which is important, for example, for use at the cardio-esophageal junction. A method for expanding the stop mechanism is disclosed.

The needle, shown in FIGS. 3A-3B, needs to be either short enough or flexible enough, to pass through the angulated entry of the biopsy channel just beyond the port below the hand controls of the flexible endoscope. In some embodiments, the needle, for flexibility, can be formed of very thin stainless steel, NiTi, or a polymer. The needle may be sufficiently flexible, in some desirable embodiments, to be used without reducing the bending section at the tip. This differentiates it from the available EUS needles, which are too stiff to be used in a conventional flexible endoscope with more than about 30 degrees of bend. A high degree of flexibility is desirable for placing stitches under the cardio-esophageal junction, for example for treating GERD. If a rigid needle (made of e.g. stainless steel) is used, the length of the needle will preferably be about 1 cm or shorter. A very short needle could be made thicker than available EUS needles without compromising the ability to negotiate bends. The diameter of the needle is preferably about 18-20 gauge. The needle can preferably be soldered, welded or otherwise attached to a structure to transmit axial force. For example, the needle can be mounted on a braided or wire-wound, hollow catheter with a PTFE or other low friction coating. Alternatively, the needle can be mounted on a suitable plastic catheter or thin-walled metal tube. The needle catheter length must be sufficient to pass through the endoscope biopsy channel and connect to a handle with enough additional working length to reach the target tissue and carry out the sewing method as described herein.

The needle can be sheathed in order to protect the biopsy channel as the needle passes through the scope. One embodiment, shown in FIGS. 3A-3B, would use a short, disposable needle sheath that is ejected as soon as the needle reaches beyond the tip of the flexible endoscope. Another embodiment, shown in FIGS. 4A-4E, would use a split protective needle sheath with an innate springiness that would spring open as the needle moves beyond the tip of the flexible endoscope. The opened split protective needle sheath would also act as a stop to control the depth of needle penetration into the tissue. The protective sheath could be metal, such as stainless steel or NiTi, or puncture resistant plastic, such as PE, PU, Nylon, and other similar materials. The sheath's functionality as a depth stop would not be affected by flexure of the endoscope. The split protective needle sheath would close automatically as the needle is withdrawn into the biopsy channel of the flexible endoscope. Other embodiments comprising a distal rather than proximal depth stop are also contemplated as such distal depth stops can be advantageous because they are not affected by flexure of the scope.

The device can preferably include a release mechanism for the T member and suture of a T-tag fastener. A highly flexible wire push rod, such as one formed of NiTi or stainless steel, could be used to eject the T member of the T-tag fastener from the distal end of the needle after it has penetrated the tissue to a predetermined depth. Hydraulic release of the T member would be another option. Alternatively, the T member of the T-tag fastener could be mounted on the end of the catheter to act as a needle for penetrating the tissue. In this embodiment, the T member can include a penetrating point at its distal end. In another embodiment, the catheter could act as the pushing rod or a coaxial pushing rod could be used to separate the T member from the catheter. The suture of the T-tag fastener could pass through the hollow catheter or outside of it.

The handle, which is connected to the needle catheter, is preferably configured to provide precise control over the movement of the needle and the pushing rod or T-tag ejector to carry out the method as described below.

The suture of the T-tag fastener can be tied using conventional methods or the T-tag fastener may optionally include a suture locking mechanism as is known in the art.

By way of example, the sewing method is described below using the embodiment of the sewing device shown in FIGS. 4A-4E.

Method steps:

The flexible endoscope is maneuvered to the target tissue.

The needle catheter is advanced through the biopsy channel of the scope.

The split protective needle sheath opens as the needle emerges from the tip of the scope.

The needle is plunged into the gastric tissue to a depth of 2-3 mm, with the open protective needle sheath acting as a stop to control the depth of needle penetration.

The pusher is advanced to eject the T member of the T-tag fastener from the distal end of the needle just beyond the serosal surface.

The needle catheter is withdrawn into the biopsy channel of the scope and the split protective needle sheath closes.

The suture is secured by tying or by pushing a suture lock onto the suture.

Optionally, the device may be configured to perform the sewing, locking and cutting of the suture in a single action. If the suture is passed through an open locking mechanism over the needle, the suture could be locked by pushing the catheter, sheath and lock forward.

While the present invention has been described herein with respect to the exemplary embodiments and the best mode for practicing the invention, it will be apparent to one of ordinary skill in the art that many modifications, improvements and subcombinations of the various embodiments, adaptations and variations can be made to the invention without departing from the spirit and scope thereof.

What is claimed is:

1. A tissue anchor deployment system, for advancing through a channel in an endoscope, comprising:
a tubular body, having a sharpened distal end;
a tissue attachment structure within the tubular body; and
a removable sheath surrounding at least the sharpened distal end, for isolating the sharpened distal end from a wall of the channel.

2. An intragastric support frame or implantation in the stomach, comprising:
at least a first and a second inflatable balloon, each having an elongate curved body with a proximal end and a distal end, at least a first and a second inflatable balloon connected together at each of the proximal and distal ends to form a support frame;
wherein the fully assembled and inflated support frame is sufficiently dimensioned to prevent passage through the pyloris.
